(19) United States
(12) Patent Application Publication
(10) Pub. No.: US 2025/0339401 A1
(43) Pub. Date: Nov. 6, 2025

(54) METHODS AND MATERIALS FOR TREATMENT OF NEISSERIA GONORRHOEAE INFECTION

(71) Applicant: Purdue Research Foundation, West Lafayette, IN (US)

(72) Inventors: Padinjaremadhom V. RAMACHANDRAN, West Lafayette, IN (US); Mohamed SELEEM, Blacksburg, VA (US)

(21) Appl. No.: 18/550,288

(22) PCT Filed: Mar. 23, 2022
(86) PCT No.: PCT/US2022/021467

§ 371 Date: Sep. 12, 2023

Related U.S. Application Data
(60) Provisional application No. 63/166,309, filed on Mar. 26, 2021.

(51) Int. Cl. A61K 31/365; A61P 31/04

(52) U.S. Cl. CPC A61K 31/365; A61P 31/04

(57) ABSTRACT

The present invention discloses series of compounds as a new antibiotic for the treatment of a subject with various infections, including infections caused by Neisseria gonorrhoeae. In particular, those compounds comprise α-methylene and α-aminomethyl lactones, lactams, iminolactones, and iminolactams, thiolactones, thionolactones, thiolactams, and thionolactams. Pharmaceutical compositions and methods for treating those infections are within the scope of this invention.

### CROSS-REFERENCE TO RELATED APPLICATIONS

The present U.S. patent application relates to and claims the priority benefit of U.S. Provisional Patent Application Ser. No. 63/166,309, filed Mar. 26, 2021, the contents of which are hereby incorporated by reference in its entirety into this disclosure.

### TECHNICAL FIELD

The present invention generally relates to compounds and methods for the treatment of a patient with a bacterial infection, particularly to α-methylene and α-aminomethyl lactones, lactams, iminolactones, and iminolactams, thiolactones, thionolactones, thiolactams, and thionolactams for the treatment of infections caused by Neisseria gonorrhoeae.

### BACKGROUND

This section introduces aspects that may help facilitate a better understanding of the disclosure. Accordingly, these statements are to be read in this light and are not to be understood as admissions about what is or is not prior art.

Gonorrhea, a sexually transmitted infection (STI), has become a noteworthy and urgent public health concern globally [1]. Each year, an estimated 78 million people are infected with gonorrhea. For almost eight decades, Gonorrhea was successfully treated/controlled by use of antimicrobials [2]. The bacterial agent responsible for gonorrhea, Neisseria gonorrhoeae (gonococcus) is evolving into an uncontrollable superbug. A high prevalence of strains of N. gonorrhoeae that is resistant to most antimicrobials, such as sulfonamides, penicillins, earlier cephalosporins, tetracyclines, macrolides, and fluoroquinolones, etc. used to treat the infection has emerged recently. The U.S. Department of Health and Human Services Centers for Disease Control and Prevention (CDC) has designated Gonorrhea as one of three urgent bacterial threats, among three prioritized levels, namely: urgent, serious, and concerning [3]. It is also included in similar antimicrobial resistance (AMR) priority lists in the UK and Canada. Major pharmaceutical companies are continually abandoning antibiotics research projects, due to non-profitability, making the situation worse [4]. Although the search for new antimicrobial drugs remains a top priority for CDC, not many antibiotics have been approved for gonorrhea. The prediction is that, if preventive measures are not undertaken, antimicrobial-resistant gonococci will soon become a public health crisis that will be a big burden to the already burgeoning health care costs. New classes of affordable and safe anti-gonorrhea drugs are desperately needed. There remains a desperate need to develop selective and cost-effective antibiotics with improved efficacy to treat gonorrhea with little or no side effects.

### SUMMARY OF THE INVENTION

The present invention generally relates to compounds useful for the treatment of an infectious disease. In some illustrative embodiments, the present invention relates to a compound having a formula

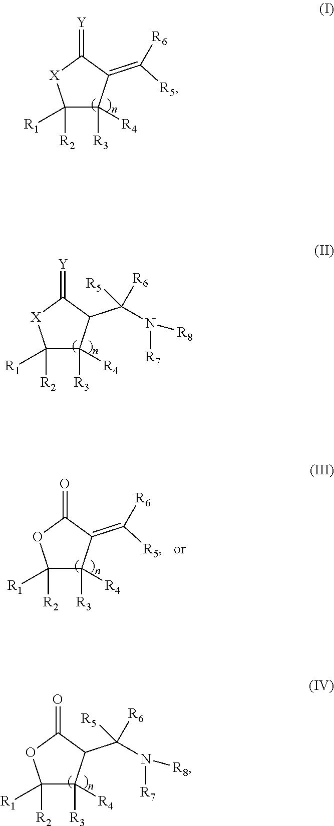

or a pharmaceutically acceptable salt thereof, wherein
X═O, NH, NR, S; Y═O, NH, NR, S; n=1, 2, 3, 4; and R₁-R₆ in the general formulas are the same or different and can be independently a hydrogen atom, a halogen atom, a hydroxyl group, and any other linear, branched, or cyclic aliphatic group containing any number of carbon (1-30) atoms. These groups can contain one or more heteroatoms, such as oxygen, sulfur, or nitrogen. Representative groups may also include but not limited to alkyl, substituted alkyl, alkenyl, substituted alkenyl, alkynyl, substituted alkynyl, aryl, substituted aryl (eg. ortho-, and/or meta-, and/or para-substituted phenyl), heteroaryl, and substituted heteroaryl. Representative substitutions include but not limited to alkoxy, alkylthio, halo, hydroxyl, phenoxy, aryloxy, cyano, isocyano, carbonyl, carboxyl, amino, amido, sulfonyl, substituted heterocyclic, etc. The amino methyl groups R₇ and R₈ can be independently a hydrogen atom, and any other linear, branched, or cyclic aliphatic groups or aryl or heteroaryl groups or substituted aryl groups. R₇ and R₈ can be part of a ring containing two or higher number carbons, such as in a aziridine, azetidine, pyrrolidine, piperidine, etc. These cycles can contain other heteroatoms, such as N or S, etc. found in morpholine, thiomorpholine, respectively. Those compounds are especially useful in the treatment of infections caused by Neisseria gonorrhoeae.

In some other embodiments, the present invention relates to a method of use of a compound or a pharmaceutically acceptable salt thereof disclosed herein in the manufacture of a medicament for treating infection in a subject.

In some other embodiments, the present invention relates to a pharmaceutical composition comprising a compound disclosed herein, together with one or more pharmaceutically acceptable diluents, excipients or carriers.

These and other features, aspects and advantages of the present invention will become better understood with reference to the following detailed description and claims.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The present invention generally relates to compounds useful for the treatment of infectious diseases. Pharmaceutical compositions and methods for treating those diseases are within the scope of this invention.

In some illustrative embodiments, the present invention relates to a method for treating a patient with an infection comprising the step of administering a therapeutically effective amount of one or more compounds of formula (I), or a pharmaceutically acceptable salt thereof, together with one or more carriers, diluents, or excipients, to a patient in need of relief from said infection:

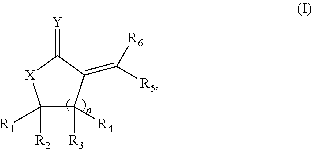

wherein
X═O, NH, NR, S; Y═O, NH, NR, S; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a method for treating a patient with an infection comprising the step of administering a therapeutically effective amount of one or more compounds of formula (I), or a pharmaceutically acceptable salt thereof, wherein said infection is caused by Neisseria gonorrhoeae.

In some illustrative embodiments, the present invention relates to a method for treating a patient with an infection comprising the step of administering a therapeutically effective amount of one or more compounds of formula (II), or a pharmaceutically acceptable salt thereof, together with one or more carriers, diluents, or excipients, to a patient in need of relief from said infection:

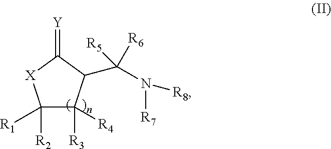

wherein
X═O, NH, NR, S; Y═O, NH, NR, S; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a method for treating a patient with an infection comprising the step of administering a therapeutically effective amount of one or more compounds of formula (II), or a pharmaceutically acceptable salt thereof, together with one or more carriers, diluents, or excipients, to a patient in need of relief from said infection, wherein said infection is caused by Neisseria gonorrhoeae.

In some illustrative embodiments, the present invention relates to a method for treating a patient with an infection comprising the step of administering a therapeutically effective amount of one or more compounds of formula (III), or a pharmaceutically acceptable salt thereof, together with one or more carriers, diluents, or excipients, to a patient in need of relief from said infection:

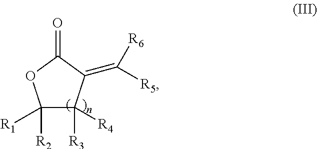

wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a method for treating a patient with an infection comprising the step of administering a therapeutically effective amount of one or more compounds of formula (III), or a pharmaceutically acceptable salt thereof, together with one or more carriers, diluents, or excipients, to a patient in need of relief from said infection, wherein said infection is caused by Neisseria gonorrhoeae.

In some illustrative embodiments, the present invention relates to a method for treating a patient with an infection comprising the step of administering a therapeutically effective amount of one or more compounds of formula (IV), or a pharmaceutically acceptable salt thereof, together with one or more carriers, diluents, or excipients, to a patient in need of relief from said infection:

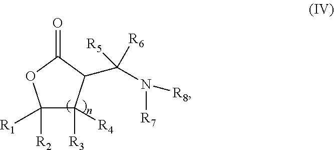

wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a method for treating a patient with an infection comprising the step of administering a therapeutically effective amount of one or more compounds of formula (IV), or a pharmaceutically acceptable salt thereof, together with one or more carriers, diluents, or excipients, to a patient in need of relief from said infection, wherein said infection is caused by Neisseria gonorrhoeae.

In some illustrative embodiments, the present invention relates to a method for treating a patient with an infection comprising the step of administering a therapeutically effective amount of compound of formula

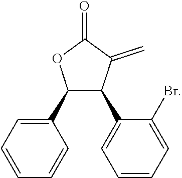 [SMILES: C=C1C(=O)O[C@H](c2ccccc2)[C@@H]1c1ccccc1Br]

In some illustrative embodiments, the present invention relates to a method for treating a patient with an infection comprising the step of administering a therapeutically effective amount of compound of formula

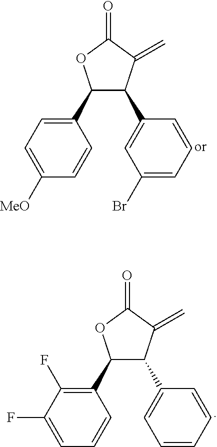 [SMILES: C=C1C(=O)O[C@H](c2ccc(OC)cc2)[C@@H]1c1cccc(Br)c1 ; C=C1C(=O)O[C@H](c2cccc(F)c2F)[C@H]1c1ccccc1]

In some illustrative embodiments, the present invention relates to a compound for treating a patient with an infection having the formula

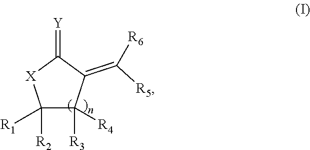

or a pharmaceutically acceptable salt thereof, wherein
X═O, NH, NR, S; Y═O, NH, NR, S; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound for treating a patient with an infection having the formula

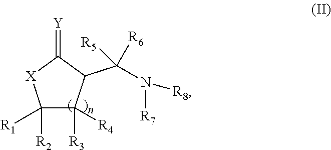

or a pharmaceutically acceptable salt thereof, wherein
X═O, NH, NR, S; Y═O, NH, NR, S; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a compound for treating a patient with an infection having the formula

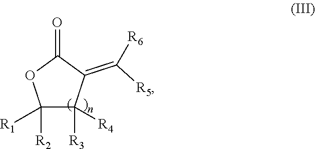

or a pharmaceutically acceptable salt thereof, wherein n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound for treating a patient with an infection having the formula

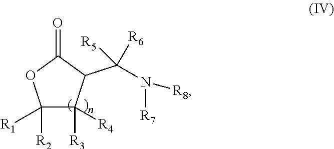

or a pharmaceutically acceptable salt thereof, wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

In some illustrative embodiments, the present invention relates to a method for treating a patient with an infection comprising the step of administering a therapeutically effective amount of compound of formula

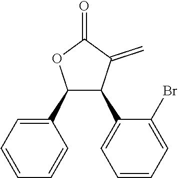 [SMILES: C=C1C(=O)O[C@H](c2ccccc2)[C@@H]1c1ccccc1Br]

or a pharmaceutically acceptable salt thereof.

In some illustrative embodiments, the present invention relates to a compound for treating a patient with an infection comprising the step of administering a therapeutically effective amount of compound of formula

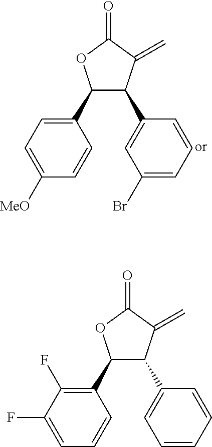 [SMILES: C=C1C(=O)O[C@H](c2ccc(OC)cc2)[C@@H]1c1cccc(Br)c1 ; C=C1C(=O)O[C@H](c2cccc(F)c2F)[C@H]1c1ccccc1]

or a pharmaceutically acceptable salt thereof.

In some illustrative embodiments, the present invention relates to a pharmaceutical composition comprising one or more compounds disclosed herein, together with one or more pharmaceutically acceptable diluents, excipients or carriers.

In some illustrative embodiments, the present invention relates to a pharmaceutical composition comprising nanoparticles of one or more compounds of disclosed herein, together with one or more diluents, excipients or carriers.

In some illustrative embodiments, the present invention relates to the use of one or more compounds disclosed herein or a pharmaceutically acceptable salt thereof in the manufacture of a medicament for treating Neisseria gonorrhoeae infection in a subject.

In some other embodiments, the present invention relates to a method of use of a compound or a pharmaceutically acceptable salt thereof disclosed herein in the manufacture of a medicament for treating infection in a subject.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2.2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₆), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N (group) 3 wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo.” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted.” or “optional substituents,” as used herein, means that the groups in question are either unsubstituted or substituted with one or more of the substituents specified. When the groups in question are substituted with more than one substituent, the substituents may be the same or different. When using the terms “independently,” “independently are.” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans isomers, diastereomers, enantiomers, and E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds and chiral carbons, while including mixtures of geometry at one or more other double bonds and chiral carbons.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th cd. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile nonaqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The pharmaceutical research and development pipeline for gonorrhoea is relatively empty, with only 3 new candidate drugs in various stages of clinical development [5]: solithromycin (by Cempra Inc.), for which a phase III trial has recently been completed; zoliflodacin (by Entasis Therapeutics), which has completed a phase II trial; and Gepotidacin (GlaxoSmithKline), which has also completed a phase II trial.

A number of naturally occurring chemopreventive and cytoprotective agents contain electrophilic α, β-unsaturated carbonyl groups (FIG. 4, circled) [13]. Naturally occurring compounds containing alpha-methylene gamma-butyrolactones (AMGBL), such as sesquiterpene lactones, have attracted interest from investigators due to their anti-cancer properties [14]. Parthenolide is an example of such a lactone that has attracted considerable attention recently [15]. Dimethylaminoparthenolide (DMAPT), readily accessed by aminating parthenolide with dimethylamine is believed to undergo deamination to the bio-active AMGBL, parthenolide [16]. Recently Crooks and co-workers reported the preparation and anti-cancer properties of a series of aminoparthenolides [17]. Synthetic AMGBLs and α-aminomethyl-γ-butyrolactones (AAGBLs) have also been shown to possess potent anti-cancer properties [18]. However, there has been scarce reports [19, 20] of the examination of natural AMGBLs as anti-bacterials and no report of the examination of synthetic AMGBLs.

This invention is based on the expectation that synthesis of α-methylenelactones and α-alkylaminolactones, particularly the γ-lactones will provide a novel class of molecules for the treatment of bacterial infection.

The present invention relates to the use of β, γ-disubstituted α-methylene-γ-hydroxy esters (AMGHEs, A), β, γ-disubstituted α-methylenelactones (AMGBLs, B) and β,γ-disubstituted-α-aminomethyllactones (AAMLAs, C), lactams, thiolactones, thionolactones, thiolactams, iminolactones, and iminolactams with the general structural formula shown below, for treating the sexually transmitted infection, gonorrhoeae. Currently, this invention focuses particularly on α- and β-disubstituted α-methylene-γ-hydroxy esters (AMGHEs), α-methylene-β- or γ- or β, γ-substituted-γ-lactones (AMGBLs) and α-aminomethyl-β- or γ- or β, γ-substituted-γ-lactones (AAMLAs) as effective, selective, and potent drug candidates for treating gonorrhoeae.

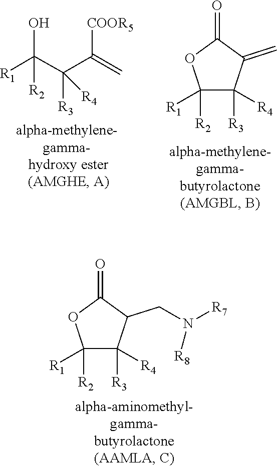

A series of γ-hydroxy-α-methylene esters of the general structural formula V were prepared according to published procedures [21-25]. They were converted to the corresponding AMGBL (general structural formula VII) as described earlier. Molecules of formula VII were converted to the corresponding α-aminomethyllactones (general structural formula VI) as described [26]. Molecules of formula V were directly converted to molecules VI as previously described [27].

The synthesis is shown in scheme 1.

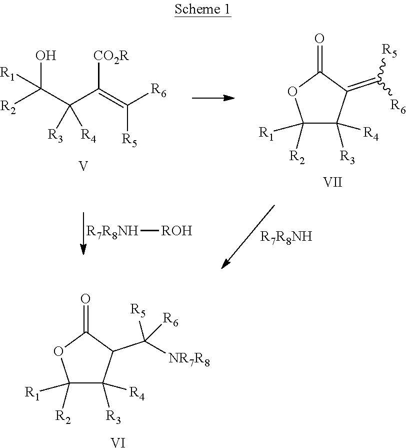

Representative procedures for the synthesis of compounds of formulae V, VI, and VII are given below.

1. General procedure for preparation of cis-γ-hydroxyester (compound formula V) [21].

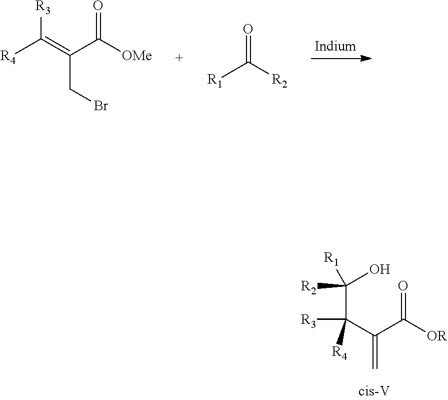

An appropriate aldehyde or ketone (1.5 equiv) were added to a solution of alkyl bromomethacrylate (1.0 eq) in a mixture of THF and water. The solution was rapidly stirred at room temperature and indium powder (1.2 equiv) was added in one portion. The suspension was stirred until the reaction was complete as judged by TLC analysis. The mixture was partitioned between water and ethyl acetate, and the organic layer was removed. The crude product was extracted from the aqueous layer one further time with ethyl acetate. The combined organic layers were washed with water then brine, then was dried over Na₂SO₄. After filtration, the product was purified via flash column chromatography to yield the desired cis-γ-hydroxyester.

2. General procedure for the preparation of trans-lactone (compound formula VII).

(i) General procedure for the preparation of ‘crotyl’ boronate [22].

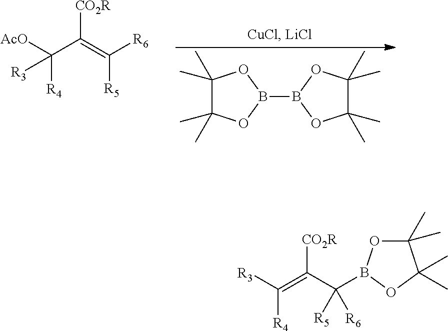

A mixture of CuCl (1.3 equiv) and LiCl (1.3 equiv) in DMF was stirred under N₂ for 1 h at 25° C. and the diboronate (Bis(pinacolato)diboron) (1.3 equiv) dissolved in DMF was added to the reaction mixture. KOAc (1.3 equiv) was added to the reaction mixture, followed by the addition of the allylic acetate (1.0 equiv) dissolved in DMF. The reaction mixture was further stirred for 3-5 h. The reaction mixture was then quenched with water and extracted with ether. The combined organic layers were dried over MgSO₄, concentrated under vacuum, and purified by column chromatography to obtain crotylboronates.

(ii) General procedure for the preparation of trans-lactones [23].

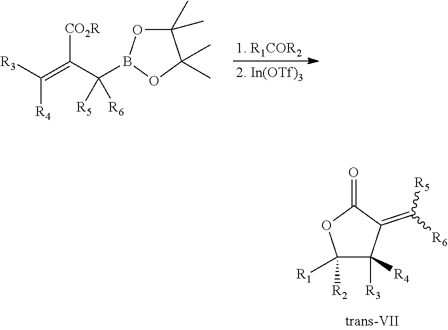

Crotylboronate (1 equiv) was dissolved in toluene and aldehyde (1.2 equiv) was added. The reaction mixture was refluxed for 8-12 h until the completion of the reaction as was monitored by ¹¹B NMR spectroscopy. The reaction mixture was then cooled to room temperature and 20% In(OTf)₃ was added, along with 20% aldehyde and stirred for 2-4 h. The mixture was washed with water and the product was extracted with ether (3×5 mL). The combined organic layer was washed with brine, dried over anhydrous MgSO₄, and concentrated in vacuo. The crude product was purified by column chromatography to obtain the trans-lactones.

3. Alternative procedure for the preparation of trans-lactone (compound formula VII) [24].

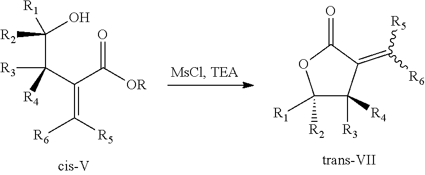

Triethylamine (1.1 equiv) was added to a solution of γ-hydroxyester (1.0 equiv) in dichloromethane (DCM). Methanesulfonyl chloride (1.1 equiv) was added drop-wise, and the reaction mixture was stirred until judged complete by TLC analysis. The reaction mixture was quenched with saturated, aqueous ammonium sulfate, then was extracted with DCM. After concentrating the combined organic layers in vacuo, the product was purified via column chromatography to obtain the trans-lactone.

4. General procedure for the preparation of cis-lactone (compound formula VII) [25].

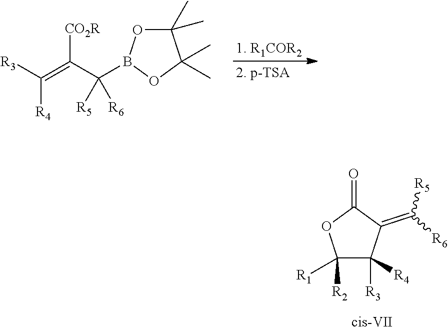

Crotylboronate (1 equiv) was dissolved in toluene and aldehyde (1.2 equiv) was added. The reaction mixture was refluxed for 8-12 h until the completion of the reaction as was monitored by ¹¹B NMR spectroscopy. The reaction mixture was then cooled to room temperature and 20% p-TSA was added, along with 20% aldehyde and stirred for 4-5 h. The mixture was washed with water and the product was extracted with ether. The combined organic layer was washed with brine, dried over anhydrous MgSO₄, and concentrated in vacuo. The crude product was purified by column chromatography to obtain the cis-lactones.

5. Alternative procedure for the preparation of cis-lactones [20].

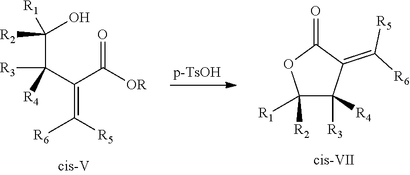

To a solution of cis-γ-hydroxyester (1.0 equiv) in DCM was added p-toluenesulfonic acid (0.1 equiv). The solution was stirred until judged as complete by TLC. After partitioning between water and dichloromethane, the organic layer was dried over Na₂SO₄. The crude product was purified by column chromatography to obtain the desired cis-lactone.

6. General procedure for the preparation of the aminolactone via conjugate addition to VII (compound formula VI) [26].

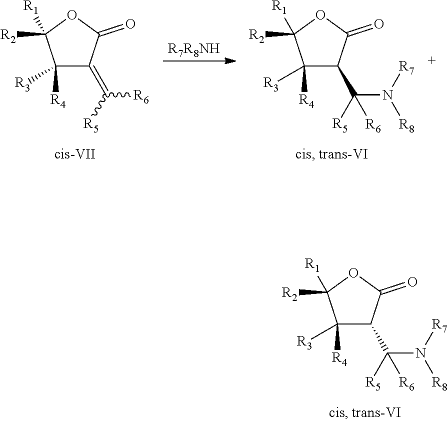

To a solution of cis-α-methylenelactone (1.0 equiv) in DCM was added N,N-dialkylamine (3.0 equiv) in one portion at room temperature. The solution was rapidly stirred until the reaction was complete as judged by TLC analysis. The crude mixture was directly concentrated in vacuo to remove the solvent and excess amine. If deemed necessary, the crude reaction mixture was purified by flash column chromatography with amine additive to yield the desired aminolactone.

7. General procedure for aminolactonization. Direct synthesis of aminolactones (compound formula VI) from γ-hydroxyesters (compound formula V) [27].

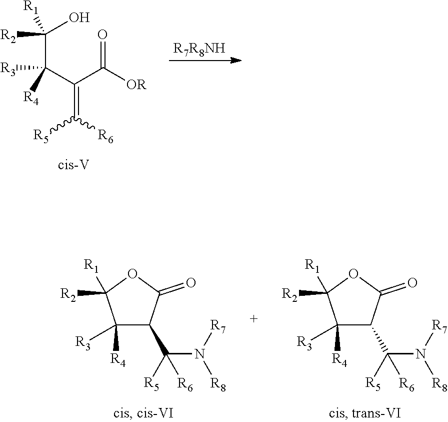

To a solution of cis-γ-hydroxyester (1.0 equiv) in dichlormethane was added N,N-dialkylamine (3.0 equiv) in one portion at room temperature. The solution was rapidly stirred until the reaction was complete as judged by TLC analysis. The crude mixture was directly concentrated in vacuo to remove the solvent and excess amine. If deemed necessary, the crude reaction mixture was purified by flash column chromatography with amine additive to yield the desired aminolactone.

### Bio-Assay

The current invention provides a method for controlling Neisseria Gonorrhoeae bacterial cell growth by arresting the cell cycle or causing cell death. The invention involves treating a cell with a compound with the general structural formula I, II, III, or IV, or an organic or inorganic salt, solvate, or hydrate thereof.

Although the molecules have only been tested with cells in vitro, this invention can be applied to cells n vivo, i.e human/animal suffering from Neisseria Gonorrhoeae.

Table 1 shows the detailed structures of those 125 compounds disclosed in this application. Initial screening of compounds #1-125 against clinically relevant Neisseria gonorrhoeae isolate 191 (Tables 2 and 3).

Table 2. Anti-bacterial activities (Minimum inhibitory concentration. MIC) of tested 125 compounds and control drugs against Neisseria gonorrhoease isolate 191.

| Compd# | MIC | Compd# | MIC | Compd# | MIC | Compd # | MIC |
|---|---|---|---|---|---|---|---|
| 1 | 16 | 20 | 32 | 39 | 16 | 60 | 8 |
| 2 | 32 | 21 | 16 | 40 | 16 | 61 | >128 |
| 3 | 1 | 22 | 8 | 41 | 32 | 62 | 32 |
| 4 | 4 | 23 | 32 | 42 | 32 | 63 | >128 |
| 5 | 16 | 24 | 32 | 43 | 16 | 64 | 64 |
| 6 | 128 | 25 | >128 | 44 | 64 | 65 | >128 |
| 7 | 32 | 26 | >128 | 45 | 32 | 66 | 32 |
| 8 | 32 | 27 | 32 | 46 | 32 | 67 | 64 |
| 9 | >128 | 28 | 32 | 47 | 128 | 68 | 32 |
| 10 | 8 | 29 | 32 | 49 | 64 | 69 | >128 |
| 11 | 8 | 30 | 128 | 50 | 32 | 70 | 8 |
| 12 | 64 | 31 | 16 | 51 | >128 | 71 | 32 |
| 13 | 32 | 32 | 32 | 52 | >128 | 72 | 8 |
| 14 | >128 | 33 | 32 | 53 | 1 | 74 | 8 |
| 15 | >128 | 34 | >128 | 54 | 64 | 75 | 32 |
| 16 | >128 | 35 | >128 | 55 | 8 | 76 | 64 |
| 17 | 128 | 36 | 128 | 56 | >128 | 77 | 32 |
| 18 | 128 | 37 | 32 | 57 | >128 | 78 | 16 |
| 19 | <128 | 38 | 16 | 59 | 16 | 79 | 16 |

| Compd# | MIC |
|---|---|
| 80 | >128 |
| 81 | 128 |
| 82 | 128 |
| 83 | 128 |
| 84 | 128 |
| 85 | 32 |
| 86 | 128 |
| 87 | 32 |
| 88 | 32 |
| 89 | 32 |
| 90 | 8 |
| 91 | >128 |
| 92 | >128 |
| 93 | 128 |
| 94 | 32 |
| 95 | >128 |
| 96 | >128 |
| 97 | 8 |
| 98 | 64 |
| 99 | 32 |
| 100 | >128 |
| 101 | >128 |
| 102 | 32 |
| 103 | 16 |
| 104 | 32 |
| 105 | 16 |
| 106 | >128 |
| 107 | 64 |
| 108 | >128 |
| 110 | 8 |
| 111 | 32 |
| 112 | >128 |
| 113 | 32 |
| 114 | >128 |
| 115 | 32 |
| 116 | >128 |
| 117 | 32 |
| 118 | 128 |
| 120 | 128 |
| 121 | 64 |
| 122 | 32 |
| 123 | 64 |
| 124 | 32 |
| 125 | 32 |
| Cefixime | 0.06 |
| Azithromycin | >128 |
| Tetracycline | 1 |

Table 2. Anti-bacterial activities (Minimum inhibitory concentration. MIC) of tested 125 compounds and control drugs against Neisseria gonorrhoease isolate 191 (Continued).

Table 3: The minimum inhibitory concentration (MIC in μg/mL) of the active compounds and control drugs (Azithromycin. Cefixime, and Tetracycline) initially screened against a panel of Neisseria gonorrhoeae strains.

| Compounds/ | Neisseria | Neisseria | Neisseria | Neisseria | Neisseria | Neisseria | Neisseria |
|---|---|---|---|---|---|---|---|
| control | gonorrhoeae | gonorrhoeae | gonorrhoeae | gonorrhoeae | gonorrhoeae | gonorrhoeae | gonorrhoeae |
| Antibiotics | 194 | 172 | 188 | 178 | 175 | 171 | 170 |
| 3 | 1 | 1 | 4 | 2 | 4 | 4 | 1 |
| 4 | 8 | 16 | 4 | 8 | 4 | 8 | 8 |
| 10 | 4 | 4 | 8 | 4 | 4 | 1 | 1 |
| 11 | 8 | 4 | 16 | 16 | 16 | 8 | 16 |
| 53 | 0.5 | 0.5 | 0.5 | 0.25 | 1 | 1 | 0.5 |
| 55 | 8 | 16 | 8 | 16 | 4 | 16 | 16 |
| 60 | 4 | 4 | 8 | 16 | 4 | 8 | 8 |
| 70 | 8 | 8 | 8 | 32 | 16 | 16 | 32 |
| 72 | 8 | 16 | 16 | 32 | 16 | 16 | 32 |
| 74 | 8 | 16 | 8 | 16 | 16 | 16 | 16 |
| 90 | 4 | 8 | 16 | 16 | 8 | 4 | 4 |
| 97 | 4 | 4 | 4 | 8 | 8 | 16 | 4 |
| 110 | 4 | 8 | 8 | 16 | 16 | 8 | 4 |
| Azithromycin | 1 | >128 | 2 | 1 | 16 | 0.25 | 1 |
| Cefixime | 1 | 0.06 | 0.25 | 0.25 | 0.03 | 0.25 | 0.25 |
| Tetracycline | 1 | 1 | 2 | 16 | 1 | 2 | 4 |

| Compounds/ | Neisseria | Neisseria | Neisseria | Neisseria | Neisseria | Neisseria |
|---|---|---|---|---|---|---|
| control | gonorrhoeae | gonorrhoeae | gonorrhoeae | gonorrhoeae | gonorrhoeae | gonorrhoeae |
| Antibiotics | 169 | 167 | 166 | 165 | 202 | 214 |
| 3 | 1 | 1 | 2 | 8 | 1 | 1 |
| 4 | 8 | 2 | 4 | 16 | 16 | 8 |
| 10 | 4 | 4 | 1 | 4 | 8 | 8 |
| 11 | 8 | 16 | 2 | 16 | 8 | 8 |
| 53 | 0.25 | 1 | 2 | 1 | 1 | 1 |
| 55 | 16 | 4 | 16 | 16 | 4 | 4 |
| 60 | 16 | 8 | 4 | 16 | 8 | 4 |
| 70 | 32 | 8 | 8 | 32 | 16 | 16 |
| 72 | 16 | 16 | 32 | 32 | 16 | 16 |
| 74 | 16 | 16 | 32 | 32 | 16 | 8 |
| 90 | 8 | 8 | 8 | 16 | 4 | 4 |
| 97 | 16 | 8 | 8 | 32 | 8 | 4 |
| 110 | 4 | 4 | 16 | 16 | 4 | 4 |
| Azithromycin | 1 | 16 | 2 | 1 | 32 | 0.5 |
| Cefixime | 0.25 | 0.06 | 0.5 | 0.25 | 0.03 | 0.25 |
| Tetracycline | 4 | 1 | 2 | 8 | 1 | 4 |

The minimum inhibitory concentrations (MICs) of the tested compounds and control drugs; Azithromycin, Cefixime and Tetracycline (antibiotics) were determined using the broth microdilution method as described in previous reports with minor modifications against clinically-relevant Neisseria gonorrhea strains. Strains were grown on Brucella broth supplemented with yeast extract, neopeptone, hematin, pyridoxal and NAD at 37° C. for 24 hours in presence of 5% CO₂. Then, bacteria were diluted in Brucella broth supplemented with yeast extract, neopeptone, hematin, pyridoxal and NAD to achieve a bacterial concentration of about 1×10⁶ CFU/mL. Compounds and control drugs were added in the first row of the 96-well plates and serially diluted with media containing bacteria. Plates were then, incubated aerobically at 37° C. in the presence of 5% CO₂ for 24 hours. MICs reported in Table 2 are those of the compounds and control drugs that could completely inhibit the visual growth of bacteria.

Next, we tested the most potent compounds against a panel of clinical N. gonorrhoeae strains to assess their activity and the results are shown in Table 3.

Analysis of Results: Compound 53 exhibited the most potent activity against a panel of Neisseria gonorrhoeae strains with MIC value ranging from 0.25 to 2 μg/mL; Compounds 3 and 10) exhibited moderate activity against N. gonorrhoeae with MIC values ranging from 1 to 8 μg/mL; Compounds 53, 3, and 10 are the most promising and interesting compounds exhibiting the most potent activity amongst the tested strains.

Those skilled in the art will recognize that numerous modifications can be made to the specific implementations described above. The implementations should not be limited to the particular limitations described. Other implementations may be possible.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. Unemo, M.; Shaferb, W. M. Antimicrobial Resistance in Neisseria gonorrhoeae in the 21st Century: Past, Evolution, and Future. Clin. Microbio. Rev. 2014, 27, 587.
2. Centers for Disease Control and Prevention: STD Surveillance 2017. https://www.cdc.gov/std/stats17/default.htm
3. CDC, Antibiotic Resistance Threats in the United States, 2013. Centers for Disease Control and Prevention. p. 1-114. 2013.
4. Editorial, Wanted: a reward for antibiotic development. Nat Biotechnol, 2018. 36 (7): p. 555.
5. WHO news release. https://www.who.int/news-room/detail/Jul. 7, 2017-antibiotic-resistant-gonorrhoea-on-the-rise-new-drugs-needed
6. Kim, K. H.; Lee, H. S.; Kim, S. H.; Lee, K. Y.; Lee, J-E; Kim, J. N.Expedient One-Pot Synthesis of γ-Hydroxybutenolides Starting from Baylis-Hillman Adducts: Lactonization, Isomerization, and Aerobic Oxidation of α-Methylene-γ-hydroxyester; Bull. Korean. Chem. Soc. 2009, 30, 1012.
7. Ishiyama, T.; Ahiko, T-A.; Miyaura, N. Acceleration Effect of Lewis Acid in Allylboration of Aldehydes: Catalytic, Regiospecific, Diastereospecific, and Enantioselective Synthesis of Homoallyl Alcohols. J. Am. Chem. Soc. 2002, 124, 12414.
8. Ramachandran, P. V.; Garner, G.; Pratihar, D.Synthesis of (E)- and (Z)-α-Alkylidene-γ-aryl-γ-butyrolactones via Alkenylalumination of Oxiranes Org. Lett. 2007, 9, 4753.
9. Park, B. R.; Kim, K. H.; Kim, J. N. An efficient synthesis of α-methylene-γ-butyrolactones from Baylis-Hillman adducts via an In-mediated Barbier reaction and stereoselective lactonization under MeSO2Cl/Et3N conditions. Tetrahedron Lett. 2010, 51,6568.
10. Kennedy, J. W. J.; Hall, D. G. Dramatic Rate Enhancement with Preservation of Stereospecificity in the First Metal-Catalyzed Additions of Allylboronates. J. Am. Chem. Soc. 2002, 124, 11586.
11. Neelakantan, S.; Nasim, S.; Guzman, M. I.; Jordan, C. T.; Crooks, P. A.Aminoparthnolides as novel anti-leukemic agents: Discovery of the NF-DB inhibitor, DMAPT (LC-1). Bioorg. Med. Chem. Lett. 2009, 19, 4346-4349.
12. Nasim, S.; Crooks, P. A. Antileukemic activity of aminoparthnolide analogs. Bioorg.Med. Chem. Lett. 2008, 18, 3870-3873.
13. Ramachandran, P. V.; Nicponski, D. R. Nair, H. N. G.; Gagare, P. D.; Helppi, M.; Schmidt, C. M.; Yip-Schneider, M. T., Synthetic α-(aminomethyl)-γ-butyrolactones and their anti-pancreatic cancer activities. Bioorg. Med. Chem. Lett. 2013, 23, 6911-6914
14. Ramachandran, P. V.; Nicponski, D. R., Diastereoselective Synthesis of α-(Aminomethyl)-γ-Butyrolactones via a Catalyst-free Aminolactonization. Chem. Commun. 2014, 50, 15216-15219.
15. Geers T.; Donabedian A. Comparison of broth microdilution and agar dilution for susceptibility testing of Neisseria gonorrhoeae. Antimicrobial agents and chemotherapy, 1989, p. 233.
16. Neelakantan, S.; Nasim, S.; Guzman, M. I.; Jordan, C. T.; Crooks, P. A.Aminoparthnolides as novel anti-leukemic agents: Discovery of the NF-DB inhibitor, DMAPT (LC-1). Bioorg. Med. Chem. Lett. 2009, 19, 4346-4349.
17. Nasim, S.; Crooks, P. A. Antileukemic activity of aminoparthnolide analogs. Bioorg.Med. Chem. Lett. 2008, 18, 3870-3873.
18. Ramachandran P V, Pratihar D, Nair H N, Walters M, Smith S, Yip-Schneider M T, Wu H, Schmidt CM. Tailored □-methylene □-butyrolactones and their effects on growth supression in pancreatic carcinoma cells. Bioorg.Med. Chem. Lett. 2010, 20, 6620-23.
19. Blakeman, J. P., Atkinson, P., Antimicrobial properties and possible role in host-pathogen interactions of parthenolide, a sesquiterpene lactone isolated from glands of Chrysanthemum parfhenium. Physiological Plant Pathology, 1979, 15, 183-192.
20. Park, B. K., Nakagawa, M., Hirota, A., Nakayama, M. Methylenolactocin, a novel antitumor antibiotic from penicillium sp.J. Antibiotics. 1988, 41 (6): 751-758.
21. Kim, K. H.; Lee, H. S.; Kim, S. H.; Lee, K. Y.; Lee, J-E; Kim, J. N.Expedient One-Pot Synthesis of γ-Hydroxybutenolides Starting from Baylis-Hillman Adducts: Lactonization, Isomerization, and Aerobic Oxidation of α-Methylene-γ-hydroxyester; Bull. Korean. Chem. Soc. 2009, 30, 1012.
22. Ishiyama, T.; Ahiko, T-A.; Miyaura, N. Acceleration Effect of Lewis Acid in Allylboration of Aldehydes: Catalytic, Regiospecific, Diastereospecific, and Enantioselective Synthesis of Homoallyl Alcohols. J. Am. Chem. Soc. 2002, 124, 12414.
23. Ramachandran, P. V.; Garner, G.; Pratihar, D.Synthesis of (E)- and (Z)-α-Alkylidene-γ-aryl-γ-butyrolactones via Alkenylalumination of Oxiranes Org. Lett. 2007, 9, 4753.
24. Park, B. R.; Kim, K. H.; Kim, J. N. An efficient synthesis of α-methylene-γ-butyrolactones from Baylis-Hillman adducts via an In-mediated Barbier reaction and stereoselective lactonization under MeSO2Cl/Et3N conditions. Tetrahedron Lett. 2010, 51, 6568.
25. Kennedy, J. W. J.; Hall, D. G. Dramatic Rate Enhancement with Preservation of Stereospecificity in the First Metal-Catalyzed Additions of Allylboronates. J. Am. Chem. Soc. 2002, 124, 11586.
26. Ramachandran, P. V.; Nicponski, D. R. Nair, H. N. G.; Gagare, P. D.; Helppi, M.; Schmidt, C. M.; Yip-Schneider, M. T., Synthetic α-(aminomethyl)-γ-butyrolactones and their anti-pancreatic cancer activities. Bioorg. Med. Chem. Lett. 2013, 23, 6911-6914
27. Ramachandran, P. V.; Nicponski, D. R., Diastereoselective Synthesis of α-(Aminomethyl)-γ-Butyrolactones via a Catalyst-free Aminolactonization. Chem. Commun. 2014, 50, 15216-15219.

1. A method for treating a patient with an infection comprising the step of administering a therapeutically effective amount of one or more compounds of formula (I) or formula (II), or a pharmaceutically acceptable salt of formula (I) or formula (II), together with one or more carriers, diluents, or excipients, to a patient in need of relief from said infection: 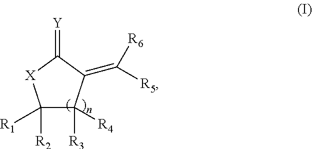
wherein
X═O, NH, NR, S; Y═O, NH, NR, S; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or 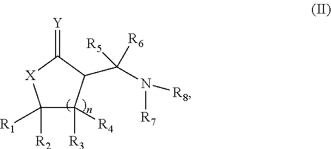
wherein
X═O, NH, NR, S; Y═O, NH, NR, S; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

2. The method of claim 1, wherein said infection is caused by Neisseria gonorrhoeae.

3. (canceled)

4. (canceled)

5. The method for treating a patient with an infection of claim 1, wherein said compound has a formula of formula (III): 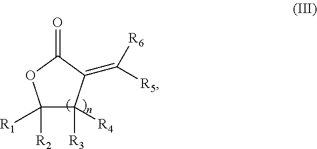
wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

6. (canceled)

7. The method of claim 5, wherein said compound has a formula of

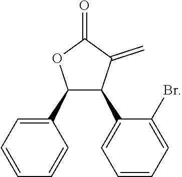 [SMILES: C=C1C(=O)O[C@H](c2ccccc2)[C@@H]1c1ccccc1Br]

8. The method of claim 5, wherein said compound has a formula of

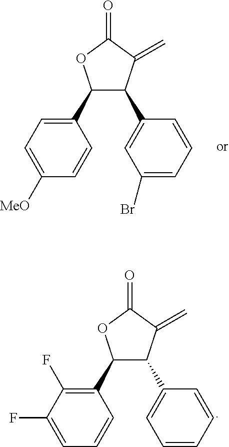 [SMILES: C=C1C(=O)O[C@H](c2ccc(OC)cc2)[C@@H]1c1cccc(Br)c1 ; C=C1C(=O)O[C@H](c2cccc(F)c2F)[C@H]1c1ccccc1]

9. The method for treating a patient with an infection of claim 1, wherein said compound has a formula of formula (IV): 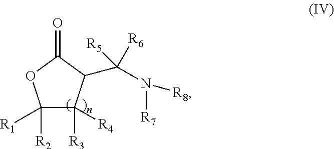
wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

10. The method of claim 7, wherein said infection is caused by Neisseria gonorrhoeae.

11. A compound for treating a patient with an infection, having the formula (I), formula (II), or a pharmaceutically acceptable salt of formula (I) or formula (II): 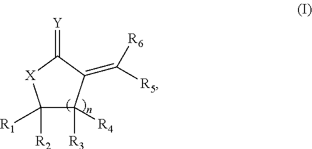
wherein
X═O, NH, NR, S; Y═O, NH, NR, S; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or 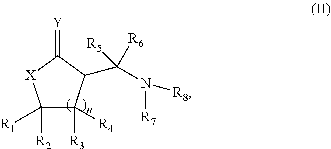
wherein
X═O, NH, NR, S; Y═O, NH, NR, S; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

12. (canceled)

13. (canceled)

14. The compound of claim 11, wherein said infection is caused by Neisseria gonorrhoeae.

15. A compound for treating a patient with an infection having formula (III) or formula (IV): 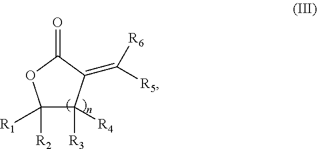
or a pharmaceutically acceptable salt thereof,
wherein
n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or 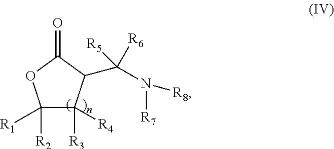
or a pharmaceutically acceptable salt thereof,
wherein
n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

16. The compound of claim 15, wherein said infection is caused by Neisseria gonorrhoeae.

17. The compound of claim 15, wherein said compound has a formula of

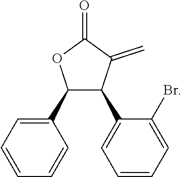 [SMILES: C=C1C(=O)O[C@H](c2ccccc2)[C@@H]1c1ccccc1Br]

18. The compound of claim 15, wherein said compound has a formula of

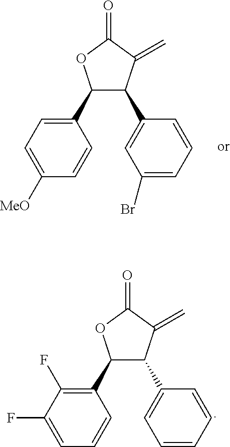 [SMILES: C=C1C(=O)O[C@H](c2ccc(OC)cc2)[C@@H]1c1cccc(Br)c1 ; C=C1C(=O)O[C@H](c2cccc(F)c2F)[C@H]1c1ccccc1]

19. (canceled)

20. (canceled)

21. A pharmaceutical composition comprising one or more compounds of formula (I) or formula (II), or a pharmaceutically acceptable salt of formula (I) or formula (II), together with one or more pharmaceutically acceptable diluents, excipients or carriers; 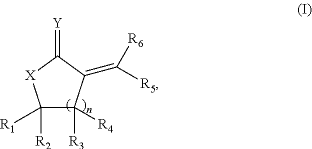
wherein
X═0, NH, NR, S; Y═0, NH, NR, S; n=1, 2, 3, 4; and
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or 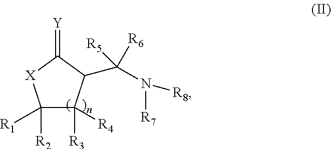
wherein
X═O, NH, NR, S; Y═O, NH, NR, S; n=1, 2, 3, 4;
R₁-R₆ are a substituent independently selected from the group consisting of hydrogen, halogen, hydroxyl, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₇-R₈ are a substituent independently selected from the group consisting of hydrogen, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; or R₇-R₈ are part of a ring system with or without one or more heteroatoms.

22. The pharmaceutical composition of claim 21, wherein the one or more compounds are nanoparticles.

23. (canceled)

24. (canceled)
